(19) United States
(12) Patent Application Publication
(10) Pub. No.: US 2022/0184033 A1
(43) Pub. Date: Jun. 16, 2022

(54) METHODS FOR PREDICTING AND REDUCING THE RISK OF PRETERM BIRTH

(71) Applicant: Kaohsiung Chang Gung Memorial Hospital, Kaohsiung City (TW)

(72) Inventors: Te-Yao Hsu, Kaohsiung City (TW); Sung-Chou Li, Kaohsiung City (TW)

(21) Appl. No.: 17/117,310

(22) Filed: Dec. 10, 2020

(51) Int. Cl. A61K 31/405; A61K 31/44; G01N 33/68; A61K 31/137

(52) U.S. Cl. CPC A61K 31/405; A61K 31/44; G01N 33/689; A61K 31/137; G01N 2333/81; G01N 2333/90283; G01N 2333/90212; G01N 2800/368; G01N 2333/908

(57) ABSTRACT

The present invention relates to a method for detecting specific proteins in amniotic fluid to predict the risk of preterm birth. The method determines different protein markers in premature amniotic fluid samples and normal amniotic fluid samples to predict the risk of preterm birth, and apply the expression levels of these protein markers to build a set of prediction models. This allows the medical staff to be prepared and greatly reduces the threat to the fetus.

### BACKGROUND

### Technical Field

The present invention relates to a method for predicting and reducing the risk of preterm birth by detecting specific proteins, and in particular, to a method for predicting preterm birth by detecting specific proteins in the amniotic fluid.

### Background

Preterm birth is defined as babies born alive between 20 weeks to less than 37 weeks of gestational age.

According to statistical data, about more than 16 million babies are born prematurely every year. Globally, preterm birth is one of the leading causes of death in children under five years old. It is estimated that there are about 1.2 million deaths related to the preterm birth each year. Moreover, many survivors facing the lifetime challenges and disabilities caused by the preterm birth, including but not limited to learning disabilities, vision disorders, auditory disorders.

The advances in neonatology over the past few decades have improved the survival rate of premature infants, however, more than 25% of premature infants suffer from at least one severe disabilities, including delayed development, chronic heart and lung diseases, brain diseases such as cerebral palsy, deafness or blindness, and the like.

The treatment of high-risk pregnancy involves preventive treatment and close monitoring to reduce the premature birth rate. However, in most cases, classifying pregnancy as high-risk based on past medical history or clinical examination can only predict a small percent of the high-risk pregnancy as preterm birth. Most preterm births cannot be identified and diagnosed at the early stage of pregnancy. Therefore, early medical interventions and treatments cannot be used in those high risk cases.

The pathogenesis or cause of preterm birth is still unclear, but if a specific method can be used to accurately predict preterm birth, it can help patients and medical staff to make the necessary preparations to reduce the health risks of newborn.

Currently, there are two evaluation methods used in clinical setting for predicting the risk of preterm birth. One is fetal fibronectin (fFN) test, by detecting fFN in the vaginal fluid, and the other test is measurement of cervical length.

Elevated fFN in vaginal fluids between 22 and 35 weeks of pregnancy has been associated with an increased risk of preterm labor and delivery. The fFN test is usually used for pregnant women with symptoms of preterm birth, such as uterine contractions, vaginal bleeding, back pain, increased vaginal discharge, and hypogastric spasm. A positive fFN result is not very good at predicting whether a woman is experiencing preterm labor and delivery. However, a negative fFN result is highly predictive that preterm delivery will not occur within the next 2 weeks.

The cervical length is 4 cm before 22 weeks of pregnancy; 3.5 cm at 22 weeks to 32 weeks of pregnancy; and reduced to 3 cm after 32 weeks. When the cervical length is shorter than 2.5 cm, there is a risk of preterm birth. Although the risk of preterm birth may be predicted by measuring cervical length, one of the drawbacks is the cervical length variation between individuals.

By combining the cervical length with fFN test, the accuracy in predicting preterm birth may be improved. However, such combination is still insufficient to accurately predict preterm birth. There is still an unmet need for predicting and/or preventing the risk of preterm birth. The present invention addresses these and other needs.

### SUMMARY

An object of the present invention is to provide methods to accurately predicting preterm birth and evaluating the risk of preterm birth in an individual, comprising the step of detect the levels of specific proteins in the amniotic fluid sample the individual to evaluate whether there is a risk of preterm birth.

The method for evaluating the risk of preterm birth in an individual provided in the present invention may be used to determine the risk or probability of preterm birth in an asymptomatic or symptomatic individual. In an embodiment, the individual is an asymptomatic individual.

The specific proteins are specific proteins existed in both normal amniotic fluid samples and premature amniotic fluid samples. The contents of the specific proteins are different in the normal amniotic fluid samples and the premature amniotic fluid samples, so that they may be used as protein markers for evaluating whether there is a risk of preterm birth.

In an embodiment, there are four proteins in amniotic fluid that may be used as protein markers for evaluating whether there is a risk of preterm birth, that is, myeloperoxidase (MPO), lactotransferrin (LTF), superoxide dismutase 2 (SOD2), and glutathione-disulfide reductase (GSR). A higher expression level of at least one of the following proteins in the amniotic fluid, relative to the expression levels of the corresponding protein in an amniotic fluid sample that is not at the risk of preterm birth, is indicative of the individual having the risk of preterm birth: MPO, LTF, SOD2, and GSR.

In an embodiment, the amount of the amniotic fluid sample of the test individual used is about 8-15 mL.

In an embodiment, the protein marker for evaluating whether there is a risk of preterm birth is LTF or SOD2.

In an embodiment, the expression level of the protein marker for evaluating the risk of preterm birth is measured by enzyme-linked immunosorbent assay (ELISA), western blotting, bicinchoninic acid assay (BCA), or Coomassie brilliant blue method, but is not limited thereto. Any detection method that may be used for measuring the expression level of protein markers for evaluating the risk of preterm birth can be used.

In an embodiment, the expression level of the protein markers for evaluating the risk of preterm birth in a plurality of premature amniotic fluid samples and a plurality of normal amniotic fluid samples, that is, the expression levels of MPO, LTF, SOD2, and GSR, are used to train the support vector machine (SVM) to establish a prediction model for evaluating the risk of preterm birth.

By using the prediction model for evaluating the risk of preterm birth, an individual who is suspected of having a risk of preterm birth is subjected to amniocentesis to extract the amniotic fluid, and the expression level of specific proteins in the amniotic fluid, that is, the expression levels of MPO, LTF, SOD2, and GSR, are measured. In an embodiment, the expression level of at least one of the above proteins is input into the prediction model for a prediction algorithm to provide a predictive result of the risk of preterm birth.

In an embodiment, if the individual is determined to have the risk of preterm birth, a therapeutic agent is administered to reduce the risk of preterm birth of the individual.

In an embodiment, the therapeutic agent for reducing the risk of preterm birth includes at least one of the following: progesterone, cervical cerclage, pessary, corticosteroid, or tocolytic agent.

In an embodiment, the tocolytic agent is a calcium channel blocker, a nonsteroidal anti-inflammatory drug (NSAID), antibiotics, a β-adrenergic receptor agonist, or magnesium sulfate.

In an embodiment, the calcium channel blocker is nifedipine.

In an embodiment, the NSAID is indomethacin.

In an embodiment, the β-adrenergic receptor agonist is terbutaline.

The present invention is characterized in that, expression levels of MPO, LTF, SOD2, and GSR in amniotic fluid are used to evaluate the risk of preterm birth, and a prediction model for evaluating the risk of preterm birth can be established according to the expression levels of the protein markers. Amniocentesis is performed in individuals who is suspected of having a risk of preterm birth. The expression levels of MPO, LTF, SOD2, and GSR in amniotic fluid is measured and then input into the prediction model to quickly and accurately predict the risk preterm birth. The entire detection procedure from performing amniocentesis to determining the risk of preterm birth only takes about two working days, which allows the medical staff to be prepared early and greatly reduces the threat to the fetus.

### BRIEF DESCRIPTION OF THE DRAWINGS

FIG. 1 shows the expression profile of 60 proteins in premature amniotic fluid samples and normal amniotic fluid samples;

FIG. 2 shows four proteins (LTF, GSR, MPO and SOD2) are differentially expressed in premature amniotic fluid samples and normal amniotic fluid samples; and

FIG. 3 shows the prediction model for evaluating the risk of preterm birth, using four proteins (LTF, GSR, MPO and SOD2) to train an SVM model.

### DETAILED DESCRIPTION

As employed above and throughout the disclosure, the following terms, unless otherwise indicated, shall be understood to have the following meanings.

As used herein, the singular forms “a,” “an,” and “the” include the plural reference unless the context clearly indicates otherwise.

The term “individual” or “subject” as used herein typically refers to a pregnant woman or a woman suspected of being pregnant.

To find proteins that can predict preterm birth, firstly, it is necessary to detect the proteins in the amniotic fluid of pregnant subjects. Then, protein biomarkers that are differentially expressed between preterm amniotic fluid and full term amniotic fluid can be used to predict the risk of preterm birth, and by applying the expression levels of the protein biomarkers to train a machine learning algorithm, a prediction model for evaluating the risk of preterm birth is established.

The establishment of the prediction model for evaluating the risk of preterm birth mainly includes the following three steps:

1. identifying differentially expressed abundant (DA) proteins from the amniotic fluid of a pregnant subject by using iTRAQ gel-free proteomics;

2. confirming the differentially expressed abundant proteins by using enzyme-linked immunosorbent assay (ELISA); and

3. establishing a prediction model for evaluating the risk of preterm birth by using a machine learning algorithm, such as support vector machine (SVM).

Once an individual is determined to have a risk of pre-term birth, a therapeutic agent can be administered to reduce the risk of preterm birth of the individual.

The therapeutic agent may be at least one of progesterone, cervical cerclage, pessary, corticosteroid, or tocolytic agent. The tocolytic agent may be at least one of a calcium channel blocker, an NSAID, antibiotics, a β-adrenergic receptor agonist, or magnesium sulfate.

Preferably, the calcium channel blocker is nifedipine.

Preferably, the NSAID is indomethacin.

Preferably, the β-adrenergic receptor agonist is terbutaline.

In summary, after the identification by iTRAQ gel-free proteomics and Partek analysis software, the resulting four proteins with significant difference between the contents in the amniotic fluid samples of PT infants and FT infants, that is, LTF, GDR, MPO, and SOD2, may be used as proteins for predicting the risk of preterm birth. Therefore, by using the foregoing prediction model for the risk of preterm birth established by using SVM with the four proteins, whether a pregnant woman to be detected has the risk of preterm birth may be accurately predicted. If there is a risk of preterm birth, the pregnant women to be detected will be treated immediately to reduce the damage to a newborn baby caused by preterm birth.

Embodiments of the present invention are illustrated by the following examples, which are not to be construed in any way as imposing limitations upon the scope thereof. During the studies described in the following examples, conventional procedures were followed, unless otherwise stated. Some of the procedures are described below for illustrative purpose.

### Example 1

Amniocentesis was performed in a pregnant subject (regardless of gestational age) in second trimester of pregnancy (16th to 18th weeks) to collect 20 mL of amniotic fluid (AF). The collected amniotic fluid sample was centrifuged to remove amniocytes, thereby obtaining a cell-free supernatant, and then the supernatant was stored at −80° C.

According to the recommendations of the American College of Obstetricians and Gynecologists, the gestational age was determined by calculating the number of days after the last menstruation. In addition, an ultrasound examination was also performed to confirm the gestational age. After an infant was born, whether the infant is full-term (FT) (the gestational age ≥37 weeks) or a pre-term (PT) (the gestational age 20 to <37 weeks) was confirmed. Finally, 36 FT amniotic fluid samples and 36 PT amniotic fluid samples were collected.

### Example 2

The proteins in the amniotic fluid samples were quantified using iTRAQ gel-free proteomics technology, which comprised the steps as follows:

Four amniotic fluid samples were identified by using iTRAQ gel-free proteomics, two of which were amniotic fluid samples from PT birth, and the other two were amniotic fluid samples from FT birth. Finally, 1,275 proteins were identified from the foregoing four amniotic fluid samples. To find the differentially abundant proteins, the following parameters were used to distinguish them from the proteins that are not differentially abundant: the error rate of protein and peptide identification <0.01, and the identified proteins contained at least ≥1 of specific peptides. After that, Partek analysis software was used to detect protein abundance. When the standard p value was set to <0.05 and the variation value was >1.25, 60 differentially abundant proteins were identified.

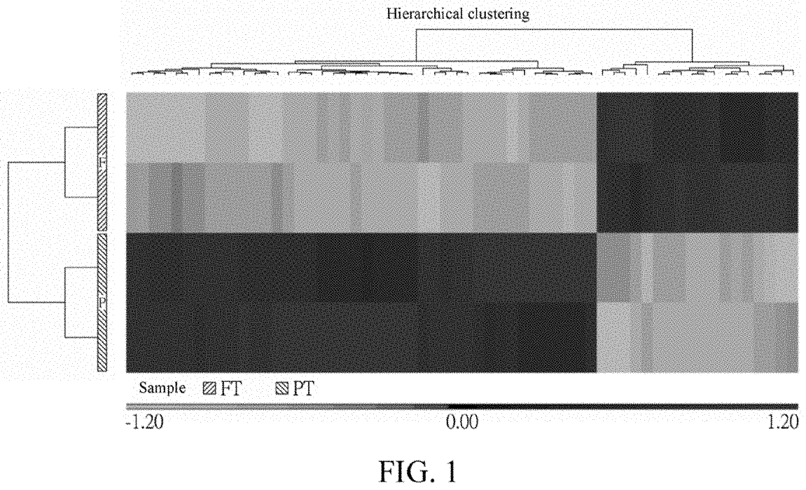

The 60 differentially abundant proteins identified by using the Partek analysis software were shown in FIG. 1. It may be seen from FIG. 1 that 75% of the proteins in FT birth samples were kept at a relatively high level, and 25% of the proteins in PT birth samples were kept at a relatively high level.

### Example 3

All the amniotic fluid samples were detected by ELISA for 6 proteins which were selected from the 60 differentially abundant proteins identified by using the Partek analysis software in Example 2, so that a t-test method may be used to confirm the reliable proteins, which can be used as candidate proteins for subsequently establishing the prediction model by using the SVM.

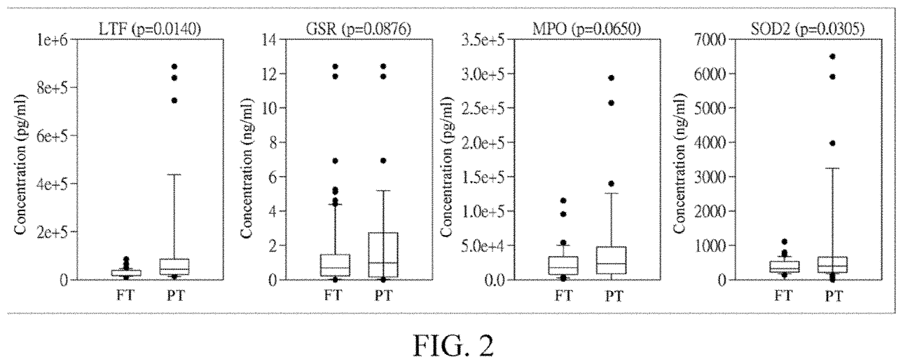

As shown in FIG. 2, in the amniotic fluid samples from 36 PT infants and the amniotic fluid samples from 36 FT infants, there are four proteins, namely, LTF, GSR, MPO, and SOD2, which have different contents in the amniotic fluid samples of PT infants and the amniotic fluid samples of FT infants, so that the four proteins may be used as the candidate proteins for the establishment of a prediction model.

### Example 4

Support vector machine (SVM) is a machine learning algorithm, which is useful in performing binary operation, such as disease group vs health group, and treatment group vs control group. After confirmed by ELISA, the data of proteins with significant difference between the contents in the amniotic fluid samples of PT infants and in the amniotic fluid samples of FT infants was input into SVM for calculation, so as to establish a prediction model for the risk of preterm birth based on the foregoing proteins.

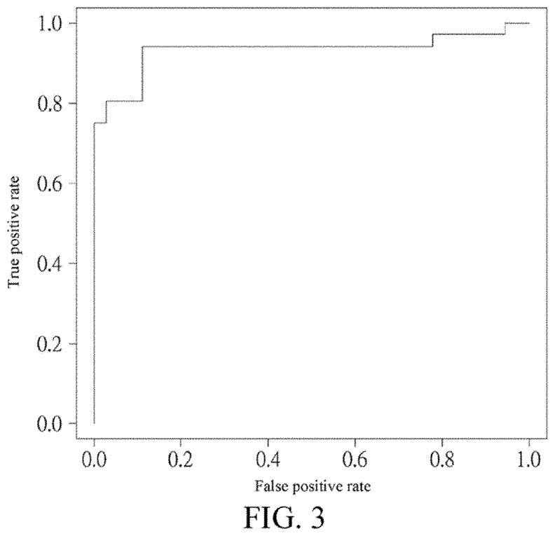

As shown in FIG. 3, when the above four proteins were used to train SVM to establish the prediction model for the risk of preterm birth, a high-performance prediction model with the auROC value=0.935 (p value=0.0001) was obtained. Therefore, this prediction model may be used to predict preterm birth in advance.

When it is impossible to confirm whether a pregnant woman is at the risk of preterm birth, the foregoing prediction model for the risk of preterm birth created by using SVM may be used to quickly determine whether the pregnant woman is at the risk of preterm birth.

The foregoing descriptions are merely preferred embodiments of the present invention, and are not intended to limit the present invention in any form. Any person of ordinary skill in the art may make some alterations or modifications according to the technical content disclosed in the present invention to obtain equivalent embodiments, without departing from the scope of the technical features of the present invention. The equivalent embodiments and any content of the technical features without departing from the present invention shall fall within the scope of the technical features of the present invention.

What is claimed is:

1. A method for evaluating and reducing the risk of preterm birth in an individual, comprising:
(a) measuring the expression of at least one of the following proteins in the amniotic fluid of the individual: myeloperoxidase (MPO), lactotransferrin (LTF), superoxide dismutase 2 (SOD2), or glutathione-disulfide reductase (GSR),
wherein, a higher expression level of at least one of the proteins in the amniotic fluid, relative to the expression levels of the corresponding protein in an amniotic fluid sample that is not at the risk of preterm birth, is indicative of the individual having the risk of preterm birth; and
(b) administering an effective amount of a therapeutic agent to reduce the risk of preterm birth of the individual.

2. The method according to claim 1, wherein the therapeutic agent comprises at least one of the following: progesterone, cervical cerclage, pessary, corticosteroid, or tocolytic agent.

3. The method according to claim 2, wherein the tocolytic agent is a calcium channel blocker, a nonsteroidal anti-inflammatory drug (NSAID), antibiotics, a β-adrenergic receptor agonist, or magnesium sulfate.

4. The method according to claim 3, wherein the calcium channel blocker is nifedipine.

5. The method according to claim 3, wherein the NSAID is indomethacin.

6. The method according to claim 3, wherein the β-adrenergic receptor agonist is terbutaline.
